(19) United States
(12) Patent Application Publication
(10) Pub. No.: US 2009/0005640 A1
(43) Pub. Date: Jan. 1, 2009

(54) METHOD AND DEVICE FOR GENERATING A COMPLETE IMAGE OF AN INNER SURFACE OF A BODY CAVITY FROM MULTIPLE INDIVIDUAL ENDOSCOPIC IMAGES

(76) Inventors: Jens Fehre, Hausen (DE); Rainer Kuth, Hochstadt (DE)

Correspondence Address: SCHIFF HARDIN, LLP;PATENT DEPARTMENT, 6600 SEARS TOWER, CHICAGO, IL, 60606-6473, US

(21) Appl. No.: 12/147,645

(22) Filed: Jun. 27, 2008

(30) Foreign Application Priority Data
Jun. 28, 2007 (DE) 10 2007 029 884.8

(51) Int. Cl. A61B 1/04

(52) U.S. Cl. 600/109

(57) ABSTRACT

In a method and a device for generation of a complete image composed from a number of individual endoscopic images of the inner surface of a body cavity of a patient, the alignment of an optical axis of an endoscope introduced into the body cavity is controlled by evaluation and comparison of the individual images acquired from different directions.

### BACKGROUND OF THE INVENTION

1. Field of the Invention

The present invention concerns a method and device to generate a complete image of an inner surface of a body cavity, the complete image being composed of a number of individual endoscopic images, using an endoscope introduced into the body cavity.

2. Description of the Prior Art

In an endoscopic examination of a body cavity of a patient, the examining physician strives to acquire the inner surface of the body cavity as completely as possible in order to avoid false-negative diagnoses (incorrect diagnoses that result in no finding) due to unacquired wall regions. However, such a complete acquisition of the inner surface of the body cavity represents a significant problem for the examining physician due to the limited image field of an endoscope and the lack of spatial depth in the presentation of the endoscopy image on a monitor, such that the risk exists that pathological regions are undetected. Although lenses known as fisheye objectives with large aperture angles up to 180° are available for image acquisition, their imaging quality is not satisfactory and the images acquired with such a fisheye objective are difficult for an observer to understand.

In order to enable optimally significant image information of the inner surface of the body cavity, it is known (for example from DE 10 2004 008 164 B3) to combine a number of individual endoscopic images acquired and stored from different positions and orientations of an endoscope into a complete image and to generate a virtual 3D model of the inner surface of the body cavities with the aid of a distance measurement system (likewise integrated into the endoscope).

A computer-assisted 3D imaging method for a wireless endoscopy apparatus (endoscopy capsule) equipped with a video camera is known from DE 103 18 205 A1. In this method the individual endoscopic images transferred to an acquisition and evaluation device are subjected to a pattern recognition algorithm in order to detect overlapping structures. In this known method the individual images are also then combined into a complete image and a 3D model.

In the known methods it is not ensured that the individual images generated with the endoscope and stored for further image processing can be combined into a gapless complete image.

### SUMMARY OF THE INVENTION

An object of the invention is to provide a method for generation of a complete image composed from a number of individual endoscopic images of the inner surface of a body cavity of a patient, with which is ensured that at least one sub-region of the inner surface is completely covered by the complete image, i.e. without gaps in the complete image. A further object of the invention is to provide a device operating according to such a method.

With regard to the method, the above object is achieved according to the invention by a method for generation of a complete image composed of a number of individual endoscopic images of the inner surface of a body cavity of a patient, wherein an optical axis of the endoscope is controlled by evaluation and comparison of the individual images acquired from different directions.

The method according to the invention ensures that the individual images are stored and available for composition of the complete image so as to gaplessly (i.e. completely) cover at least one diagnostically relevant region of the inner surface that is larger than a region acquired with an individual image.

The term “optical axis of the endoscope” is to be understood in the following as the optical axis of the imaging system utilized for endoscopic image generation in object space. This imaging system can be a video camera integrated into the endoscope tip, for example.

In an embodiment of the method, in a first step a number of individual images are acquired from predetermined different directions and stored. Any gap that occurs between adjacent individual images as well as directions respectively associated with such gaps are identified. Using these directions, an individual image is generated anew in a second step by controlling the alignment of the optical axis of the endoscope by evaluation and comparison of the individual images. The second step is repeated as often as needed until the complete image composed from the individual images no longer contains gaps.

The aforementioned number of individual images can be two successive individual images or series of successive individual images.

The alignment of the optical axis of the endoscope advantageously ensues automatically, i.e. without an intervention by the physician conducting the examination being necessary for this. As an alternative or in addition, it is possible that an optical, audio or haptic indicator is provided to the physician indicating whether, given manual control and manual image triggering, the physician has generated successive individual images with sufficient overlap for generation of a complete image formed without gaps.

The alignment of the optical axis of the endoscope can ensue by alignment of the tip of the endoscope.

In a preferred embodiment of the invention, an endoscope with a video camera, that is mounted such that it can be panned in the endoscope tip, is used to align the optical axis by such panning.

The location of the endoscope and the direction of the optical axis can additionally detected in a fixed coordinate system and stored together with the individual image determined at this location and with this direction, making it possible to link the individual endoscopic images or the complete endoscopic image with images from other imaging methods implemented during or immediately before or after the endoscopic examination.

Moreover, the distance of the endoscope tip from the inner surface of the cavity in the direction of the optical axis can be measured and stored for each individual image, and a complete 3D image is generated from the individual images and the respective associated distance. The position and the direction, a particularly intuitive representation of the body cavity, is then available for the examining physician.

The object according to the invention also is achieved by a device operating according to the above method exhibiting advantages that correspond to the advantages described with regard to the method.

### BRIEF DESCRIPTION OF THE DRAWINGS

FIG. 1 is a schematic illustration of an embodiment of a device according to the invention.

FIG. 2 is a flow chart of an exemplary embodiment for control of the optical axis of the video camera in accordance with the invention.

### DESCRIPTION OF THE PREFERRED EMBODIMENTS

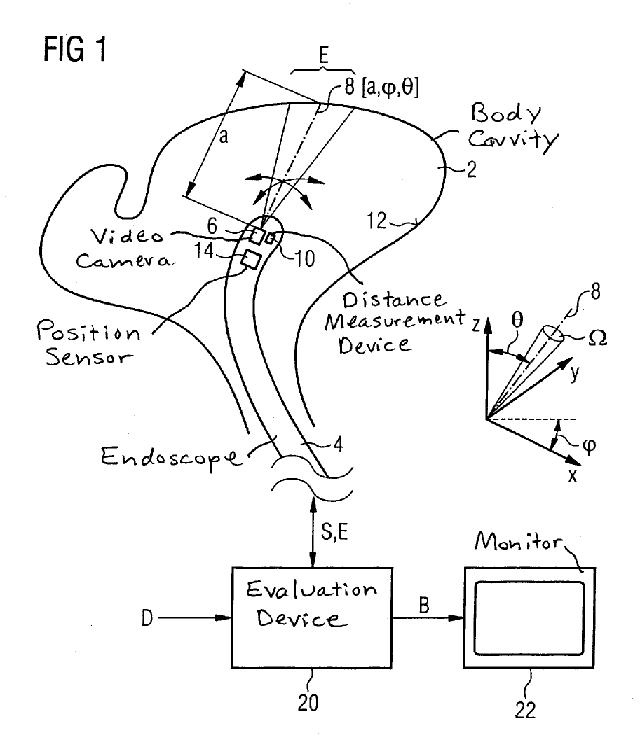

According to FIG. 1, an endoscope 4 (in the example a flexible endoscope 4) in which a video camera 6 is arranged at the distal, free end is inserted into a body cavity 2 of a patient. By pivoting the endoscope tip, the optical axis 8 of the endoscope 4 (given use of a video camera 6 installed into the endoscope tip, this is identical with the optical axis of the video camera 6) can be aligned in different directions, as this is illustrated in the Figure by two double arrows.

Deviating from the presentation of FIG. 1, the endoscope 4 can also be a rigid endoscope in which the video camera 6 is mounted such that it be panned. In a further, simplified variant, a rigid endoscope is likewise arranged in which the video camera 6 is arranged stationary such that its optical axis 8 (and therefore the optical axis of the endoscope) is askew, i.e. runs at an angle different than 0° relative to a longitudinal axis of the endoscope. The viewing direction (i.e. the direction of the optical axis of the endoscope) is then varied by rotating the endoscope.

Given use of a flexible endoscope 4 as shown in the FIG. 1, the direction of the optical axis can be pivoted on three axes perpendicular to one another with the use of multiple Bowden wires and by rotating the entire endoscope 4 around its longitudinal axis when the angle between optical axis and longitudinal axis of the endoscopy tip differs from 0°.

As an alternative, given a flexible endoscope 4 control of the video camera 6 ensues externally from the endoscope 4, for example with the use of an external magnetic field.

Moreover, a distance measurement device 10 with which it is possible to measure the distance a of the endoscope tip 4 or of the iris of the video camera 6 from the inner surface 12 of the body cavity 2 in the direction of the optical axis 8 is integrated into the endoscope tip 4. In the case of a video camera 6 arranged such that it can pan inside the endoscope 4, the distance measurement device 10 is mechanically forcibly coupled with this. Moreover, a position sensor 14 with which the position and alignment of the endoscopy tip can be detected in a fixed coordinate system x, y, z is integrated into the endoscope 4. The direction φ, θ of the optical axis 8 of the video camera 6 is also known in this fixed coordinate system x, y, z. Moreover, the solid angle acquired by the video camera 6 is plotted in the Figure with Ω.

With the aid of the video camera 6, a sub-region of the inner surface 12 is respectively rendered for different directions of the optical axis 8, and partially overlapping individual images E are generated and relayed to a control and evaluation device 20 that analyzes the individual images E (existing in digital form) and combines them into a contiguous complete image B that is rendered on a monitor 22. In order to ensure that the generated image data set B delivers a gapless complete image B of at least one section of the inner surface 12 of the body cavity, adjacent individual images are evaluated in the control and evaluation device 20 as to whether they exhibit correlating image features and overlap. In order to ensure such an overlap, control signals S with which the alignment of the optical axis 8 of the endoscope 4 is automatically controlled are generated on the basis of the result of this evaluation determined in the control and evaluation device 20. A complete image B rendering at least one region of the inner surface 12 of the body cavity 2 can be generated in this manner, which complete image B displays a surface area that is significantly larger than the field of view or image field of an individual image E and, in the ideal case, shows a complete or nearly complete 360° panoramic view of the body cavity 2.

A 3D complete image B of the inner surface 12 of the body cavity 2 can also be generated via evaluation of the distance a belonging to each individual image E acquired in the direction φ, θ and the position of the intersection point of the optical axis 8 with the inner surface 12 of the body cavity 2 that is known from this. This 3D complete image B can be inserted into a 3D data set D generated with another imaging method so that the endoscopic diagnoses can be combined with other diagnostic methods and the diagnosis reliability can be increased.

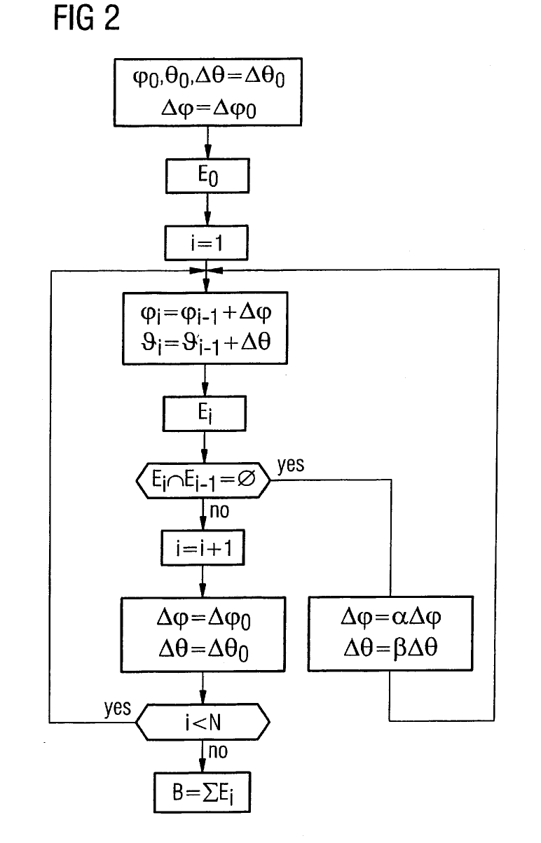

A possible workflow of the algorithm to control the alignment of the optical axis of the endoscope is exemplarily illustrated in the flow diagram according to FIG. 2. An individual image E₀ is generated in an initial position with an initial direction φ₀, θ₀ of the optical axis. An operating (running) parameter i is set to 1. Panning of the camera by the angle increments Δφ, Δθ to the new alignment φᵢ=φ₁=φ₀+Δφ, θᵢ=θ₁=θ₀+Δθ subsequently ensues by activation of the video camera. An individual image Eᵢ is newly generated with this alignment. In a next step it is checked whether the preceding individual image Eᵢ₋₁ and the subsequent adjacent individual image Eᵢ exhibit an overlap. This is symbolically illustrated in the flow diagram with the intersection set Eᵢ∩Eᵢ₋₁. If the intersection set Eᵢ∩Eᵢ₋₁ is empty (i.e. if no overlap is present), the incremental values Δφ and Δθ are respectively reduced with factors α,β<1. An individual image Eᵢ is newly generated with the aid of the new alignment φᵢ and θᵢ determined in this manner. In other words: if a missing overlap (i.e. a gap) is established, a direction belonging to this gap is identified in which a new individual image Eᵢ is generated. This direction is not necessarily the direction in which the middle of the gap lies, but rather the direction in which a new individual image Eᵢ is acquired due to the established gap. This procedure is repeated until and overlap is established. If an overlap is established, the operating parameter is increased by 1 and the incremental steps Δφ and Δθ are reset to the initial values. The method proceeds in this manner either for a predetermined number of steps N or with a variable step count N until the angle directions φ_{N} and θ_{N} correspond to the initial angle directions φ₀ and θ₀. A complete image B is now composed from the individual images Eᵢ acquired in this manner, as this is symbolically illustrated by the sum ΣEᵢ.

The example shown in FIG. 2 serves only for illustration of a possible algorithm that can in principle also run in a different manner in that, for example, more than two individual images Eᵢ are acquired from predetermined different directions in a first step (meaning that a larger angle range is covered) and in which gaps possibly situated between individual images Eᵢ as well as directions associated with these are subsequently identified via evaluation and comparison of the individual images in a composed preliminary complete image B, from which gaps and associated directions individual images are generated in a second step by controlling the alignment of the optical axis of the endoscope, wherein the second step is repeated as often as necessary until the assembled complete image B no longer exhibits gaps.

As an alternative to such an automatic control, it is also possible for the operator to manually effect the alignment of the optical axis in that he manually stores individual images, wherein after the storage of an individual image following a preceding stored individual image it is indicated to him via corresponding indicator signals that the panning movement implemented by him for the subsequent individual image was too large to enable an overlap of the individual images. The operator then receives, by acoustic, optical or haptic signals, the prompt to pan the video camera back until a corresponding overlap is established.

Although modifications and changes may be suggested by those skilled in the art, it is the intention of the inventors to embody within the patent warranted hereon all changes and modifications as reasonably and properly come within the scope of their contribution to the art.

We claim as our invention:

1. A method for generating an image of an inner surface of a body cavity, comprising the steps of:
introducing an endoscope into a body cavity of a patient, said endoscope having an optical axis;
acquiring a plurality of individual endoscopic images of an inner surface of the body cavity with the optical axis aligned in respectively different directions relative to the inner surface;
evaluating and comparing said individual images to obtain an evaluation result, and controlling alignment of said optical axis dependent on said evaluation result; and
assembling a complete image of said inner surface of said body cavity from said plurality of individual endoscopic images.

2. A method as claimed in claim 1 comprising:
storing said plurality of individual endoscopic images respectively acquired with said optical axis aligned at different directions relative to the inner surface;
evaluating the stored plurality of individual endoscopic images to identify an existence of gaps between adjacent ones of said individual endoscopic images and to identify respective directions of any such gaps;
dependent on the respective directions of said gaps identified in the evaluation of said individual endoscopic images, acquiring further individual endoscopic images with said optical axis differently aligned, and evaluating said further individual endoscopic images to identify an existence of gaps between adjacent ones of said further individual endoscopic images and to identify respective directions of said gaps between adjacent ones of said further individual endoscopic image; and
repeating acquisition of said further individual endoscopic images and evaluation thereof as to the existence of gaps until the assembled complete image is free of said gaps.

3. A method as claimed in claim 1 comprising automatically controlling alignment of said optical axis relative to said inner surface of the body cavity to acquire said individual endoscopic images from said respectively different directions.

4. A method as claimed in claim 1 comprising aligning a tip of said endoscope relative to said inner surface of the body cavity to obtain said individual endoscopic images respectively from said different directions.

5. A method as claimed in claim 1 wherein said endoscope comprises a video camera mounted at a tip of the endoscope, and panning said video camera to acquire said individual endoscopic images respectively from said different directions relative to the inner surface of the body cavity.

6. A method as claimed in claim 1 comprising, for each of said individual endoscopic images, detecting and identifying a location of a tip of the endoscope and a direction of the optical axis in a fixed coordinate system, and storing said location and direction together with the individual endoscopic image obtained at said location and direction.

7. A method as claimed in claim 6 comprising detecting and measuring a distance of the tip of the endoscope from said inner surface of the body cavity in the direction of the optical axis, and storing said distance together with each individual endoscopic image, and assembling a complete 3D image of said inner surface using the stored individual endoscopic images the respectively associated distances, positions and directions.

8. A device for generating an image of an inner surface of a body cavity, comprising:
an endoscope configured for introduction into a body cavity of a patient, said endoscope having an optical axis and said endoscope being configured to acquire a plurality of individual endoscopic images of an inner surface of the body cavity with the optical axis aligned in respectively different directions relative to the inner surface;
an evaluation unit that evaluates and compares said individual images to obtain an evaluation result, and that automatically controls alignment of said optical axis dependent on said evaluation result; and
an image computer that assembles a complete image of said inner surface of said body cavity from said plurality of individual endoscopic images.

9. A device as claimed in claim 8 comprising:
a memory that stores said plurality of individual endoscopic images respectively acquired with said optical axis aligned at different directions relative to the inner surface; and
said evaluation unit evaluating the stored plurality of individual endoscopic images to identify an existence of gaps between adjacent ones of said individual endoscopic images and to identify respective directions of any such gaps and, dependent on the respective directions of said gaps identified in the evaluation of said individual endoscopic images, causing said endoscope to acquire further individual endoscopic images with said optical axis differently aligned, and evaluating said further individual endoscopic images to identify an existence of gaps between adjacent ones of said further individual endoscopic images and to identify respective directions of said gaps between adjacent ones of said further individual endoscopic image, and causing said endoscope to repeat acquisition of said further individual endoscopic images and an evaluation unit repeating evaluation thereof as to the existence of gaps until the assembled complete image is free of said gaps.

10. A device as claimed in claim 8 wherein a tip of said endoscope is alignable relative to said inner surface of the body cavity to obtain said individual endoscopic images respectively from said different directions.

11. A device as claimed in claim 8 wherein said endoscope comprises a video camera mounted at a tip of the endoscope, and comprising a control unit that pans said video camera to acquire said individual endoscopic images respectively from said different directions relative to the inner surface of the body cavity.

12. A device as claimed in claim 1 comprising a position detection that, for each of said individual endoscopic images, detect and identifies a location of a tip of the endoscope and a direction of the optical axis in a fixed coordinate system, and a memory in which said location and direction and stored together with the individual endoscopic image obtained at said location and direction.

13. A device as claimed in claim 12 comprising a distance measuring unit that detects and measures a distance of the tip of the endoscope from said inner surface of the body cavity in the direction of the optical axis, and wherein said memory stores said distance together with each individual endoscopic image, and wherein said image computer assembles a complete 3D image of said inner surface using the stored individual endoscopic images the respectively associated distances, positions and directions.
